(19) United States
(12) Patent Application Publication
(10) Pub. No.: US 2021/0149966 A1
(43) Pub. Date: May 20, 2021

(54) SYSTEMS AND METHODS FOR PERFORMING A COMPUTER-IMPLEMENTED PRIOR ART SEARCH AND NOVEL MARKUSH LANDSCAPE

(71) Applicant: AMERICAN CHEMICAL SOCIETY, Washington, DC (US)

(72) Inventors: Todd Josef WILLS, I, Columbus, OH (US); Christopher Peter Kynnersley BADDELEY, Columbus, OH (US); Matthew Jennings McBRIDE, Camas, WA (US)

(21) Appl. No.: 16/952,970

(22) Filed: Nov. 19, 2020

Related U.S. Application Data
(60) Provisional application No. 62/938,179, filed on Nov. 20, 2019.

(51) Int. Cl. G06F 16/903; G06F 16/901; G06N 20/00

(52) U.S. Cl. CPC G06F 16/90335; G06N 20/00; G06F 16/9024

(57) ABSTRACT

In one embodiment, a computer implemented method for implementing a supervised learning engine to conduct a prior art and novel Markush landscaping search is provided. The method may include inputting a query compound into a supervised learning engine; creating, by the supervised learning engine, a query graph framework; decomposing, by the supervised learning engine, the query graph framework into at least one derivative graph node bond frameworks; adding a substituent to each of the at least one derivative graph node bond frameworks; and receiving, from the engine, an output list comprising a set of novel compounds and a set of known compounds.

### CROSS-REFERENCES TO RELATED APPLICATIONS

This application claims priority from U.S. Provisional Patent Application No. 62/938,179, filed Nov. 20, 2019, which is hereby incorporated by reference in its entirety.

### BACKGROUND

Performing prior art searches is often cumbersome and inefficient. Methods of performing prior art searches suffer from long processing times, thereby causing backlogs and delays in the patent examining process. In addition, current computerized search tools require a human to input information at one or more steps. Inefficiencies in current search methods also stem from the difficulty of quantifying textual documents, yielding sub-optimal results.

Relatedly, drafting claims that adequately define and justify the scope of an invention may not be an easy task and may often be a cumbersome process. Claim construction may be vital for properly defining a particular invention or new process.

A popular form of claim drafting, particularly in the chemical space, is the Markush claim. A Markush claim recites a list of alternatively usable members or elements. These types of claims may not only be difficult to draft but may require intensive prior art searching. If the drafter of the claims does not conduct a thorough search of the prior art, then they may draft the claims narrower than may be required by the prior art. This could result in the applicant claiming less than they may be entitled to. If the drafter of the claims does not conduct a thorough search of the prior art, then they may draft the claim broader then would be permitted under the prior art, causing the application to be rejected by the examiner. Having a properly drafted Markush claim may allow the applicant to claim broadly without the fear of having the claims rejected by the examiner.

The drafter also has to ensure the patent's written description contains enough examples (i.e., the disclosed species) to sufficiently support the scope of the Markush group (i.e., claimed genus). An adequate written description of a genus requires the specification to disclose a representative number of species falling within the scope of the genus or structural features common to the members of the genus so that one of ordinary skill in the art may visualize or recognize the members of the genus.

Thus, there exists a need for systems and methods for efficiently and accurately identifying examples within a possible Markush group.

### SUMMARY OF THE INVENTION

For some embodiments of the present invention, a computer-implemented method is provided for implementing a supervised learning engine to conduct a prior art and novel Markush landscaping search.

In one embodiment, a computer implemented system for is provided. The system may comprise a memory device storing a set of instructions and at least one processor executing the set of instructions to perform a method. The method may include a set of steps, including inputting a query compound into a supervised learning engine; creating, by the supervised learning engine, a query graph framework; decomposing, by the supervised learning engine, the query graph framework into at least one derivative graph node bond frameworks; adding a substituent to each of the at least one derivative graph node bond frameworks; and receiving, from the engine, an output list comprising a set of novel compounds and a set of known compounds.

In another embodiment, a computer-implemented method is disclosed. The method may comprise steps including: inputting a query compound into a supervised learning engine; creating, by the supervised learning engine, a query graph framework; decomposing, by the supervised learning engine, the query graph framework into at least one derivative graph node bond frameworks; adding a substituent to each of the at least one derivative graph node bond frameworks; and receiving, from the engine, an output list comprising a set of novel compounds and a set of known compounds.

In other embodiments, other systems, methods, and computer program products are provided. It is to be understood that both the foregoing general description and the following detailed description are exemplary and explanatory only, and are not restrictive of the disclosed embodiments, as claimed.

### BRIEF DESCRIPTION OF THE DRAWINGS

The accompanying drawings, which are incorporated in and constitute a part of this specification, illustrate disclosed embodiments and, together with the description, serve to explain the disclosed embodiments. In the drawings:

FIG. 1 illustrates an exemplary system for implementing a supervised learning engine to conduct a prior art and novel Markush landscaping search, in accordance with disclosed embodiments.

FIG. 2 depicts an exemplary decomposition, in accordance with disclosed embodiments.

FIG. 3 illustrates an exemplary query graph framework and derivative graph node bond framework, in accordance with disclosed embodiments.

FIG. 4 depicts exemplary derivative graph node bond frameworks, in accordance with disclosed embodiments

FIG. 5 is a flow diagram of an exemplary method of implementing a supervised learning engine to conduct a prior art and novel Markush landscaping search, in accordance with disclosed embodiments.

### DETAILED DESCRIPTION

In the following detailed description, numerous specific details are set forth in order to provide a thorough understanding of the disclosed example embodiments. However, it will be understood by those skilled in the art that the principles of the example embodiments may be practiced without every specific detail. Well-known methods, procedures, and components have not been described in detail so as not to obscure the principles of the example embodiments. Unless explicitly stated, the example methods and processes described herein are not constrained to a particular order or sequence, or constrained to a particular system configuration. Additionally, some of the described embodiments or elements thereof can occur or be performed simultaneously, at the same point in time, or concurrently.

Disclosed embodiments provide systems and methods for implementing a supervised learning engine to conduct a prior art and novel Markush landscaping search. Some embodiments disclose a supervised learning engine that is able to determine if a known Markush structure, exemplified structure, a hypothetical Markush group, or exemplified structure is in existence. Additionally, the disclosed systems and methods may be used to identify open and occupied areas surrounding a Markush group or exemplified chemical structure and any representative compounds residing in the open and occupied areas. A supervised learning engine may include using machine learning or artificial intelligence algorithms to model relationships and dependencies between a target or output variable and input data. Machine-learning models may include a supervised learning model, a neural network model, an attention network model, a generative adversarial model (GAN), a recurrent neural network (RNN) model, a deep learning model (e.g., a long short-term memory (LSTM) model), a random forest model, a convolutional neural network (CNN) model, an RNN-CNN model, an LSTM-CNN model, a temporal-CNN model, a support vector machine (SVM) model, a Density-based spatial clustering of applications with noise (DBSCAN) model, a k-means clustering model, a distribution-based clustering model, a k-medoids model, a natural-language model, and/or another machine-learning model. Models may include an ensemble model (i.e., a model comprised of a plurality of models). For example, the disclosed supervised learning engine may be implemented using the DataRobot or KNIME supervised learning system. A Markush landscape may include an identification of identified species within a genus. For example, the Markush landscape may include an indication of species within a genus that have been identified in a publicly disclosed databases, such as patent publications. Additionally or alternatively, a Markush landscape may further include an identification of species within a genus that have been claimed in a patent publication. A chemical structure may include a representation of the arrangement of chemical bonds between atoms in a molecule and may identify chemical bonds between atoms within the molecule as well as a geometric shape of the molecule. The chemical structure may uniquely identify the type of molecule.

Aspects of the disclosed embodiments may include inputting a query compound into a supervised learning engine. The supervised learning engine may be used to identify open areas surrounding a Markush group or exemplified chemical structure and any representative compounds residing in the open areas by inputting a query compound into a supervised learning engine. A query compound may include a compound of interest.

Aspects of the disclosed embodiments may include creating, by the supervised learning engine, a query graph framework. The supervised learning engine may utilize a chemical structure with a defined connection table. A connection table may include a data table that provides information for computer to generate a molecular graph. The connection table may define the atoms and connections within the compound as edges and nodes. The connection table may include additional tables, such as an atom table and a bond table. The original chemical structure provided or input may be categorized by the engine as a query compound. Once the query compound is input into the supervised learning engine, the graph framework of the query compound may be utilized within an internal database. A query graph framework may include a graph framework generated from the original query compound. A graph framework may include a data structure stored in memory representing information as nodes and relationships or connections between nodes as edges.

Aspects of the disclosed embodiments may include decomposing, by the supervised learning engine, the query graph framework into at least one derivative graph node bond framework. A query graph framework may be broken down into sections. A section may be considered to be every non-fused ring system or connecting chain and may be represented by a graph node. The engine may either add a node, subtract a node, or maintain the current number of nodes. Nodes may be vertices that represent atom locations. In some embodiments, one section may be changed at a time. A derivative graph node bond framework may include a graph framework representing the decomposed sections of the query compound. Decomposing the query graph framework may include recursively breaking down a graph framework into the smallest possible section, representing a substituent molecule or atom. A substituent may include an atom, group of atoms, molecule, or group of molecules which may replace another atom or group occupying a specified position in a molecule.

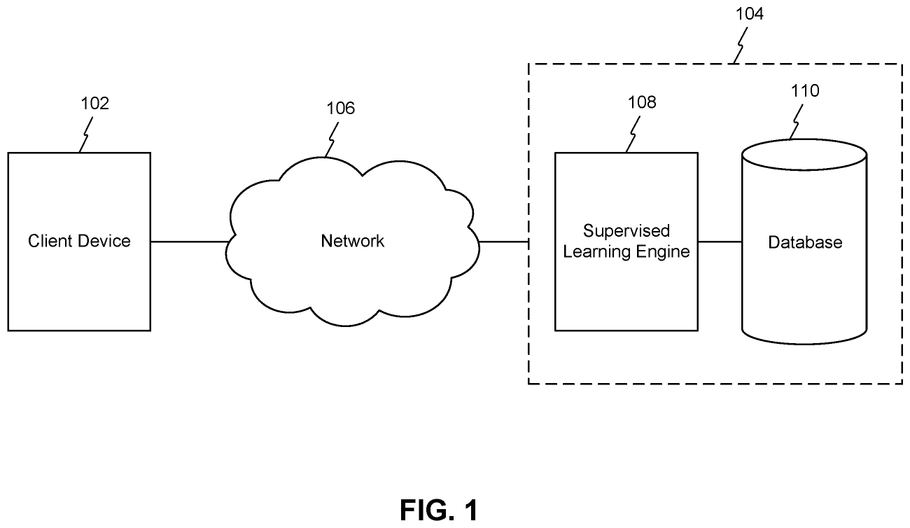

FIG. 1 illustrates an exemplary system 100 for implementing a supervised learning engine to conduct a prior art and novel Markush landscaping search. System 100 may include, for example, a client device 102 and a processing device 104 which are connected communicatively by network 106.

Network 106, in some embodiments, may be a network or networks configured to enable data communication between devices. For example, network 106 may be the Internet, an intranet, a cellular network, a satellite network, a Local Area Network (LAN), a Wide Area Network (WAN), a Metropolitan Area Network (MAN) or any other kind of network. Network 106 may be implemented using wired technologies, wireless technologies, or a combination thereof.

Processing device 104 may be a computer including a processor and memory storing instructions configured to cause the processor to perform operations Processing device 104 may include supervised learning engine 108 and database 110. In some embodiments, database 110 may be a device separate from processing device 104. In some embodiments, a database 110 may be configured to store datasets and/or one or more dataset indexes, consistent with disclosed embodiments. Database 110 may include a cloud-based database (e.g., AMAZON WEB SERVICES RELATIONAL DATABASE SERVICE) or an on-premises database. For example, a database may include an XML database, an RDBMS database, an SQL database, or a database provided by MongoDb, Redis, Couchbase, Elastic Search, Splunk, Solr, Cassandra, Amazon DynamoDb, Scylla, HBase, Neo4J, Oracle, MySQL or Microsoft SQL. Database 110 may be configured to store documents or digital representations of documents. The documents may include patent applications, patents, articles, books, articles, newspapers, magazines, journals, presentations, manuals, published scientific research, scientific literature, or any other information stored as text. Additionally or alternatively, database 110 may include information extracted from other databases. For example, a database may contain chemical compounds disclosed in patent applications, patents, articles, books, articles newspapers, magazines, journals, presentations, manuals, published scientific research, scientific literature, or other information stored as text. In some embodiments, processing device 104 may be a part of client device 102. In other embodiments, processing device 104 may be a separate computing resource.

In some embodiments, database 110 may store information in a data structure, e.g., a graph structure. Database 110 may be implemented using, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), etc. The memory drives may further include a drum, magnetic disc drive, magneto-optical drive, optical drive, redundant array of independent discs (RAID), solid-state memory devices, solid-state drives, etc.

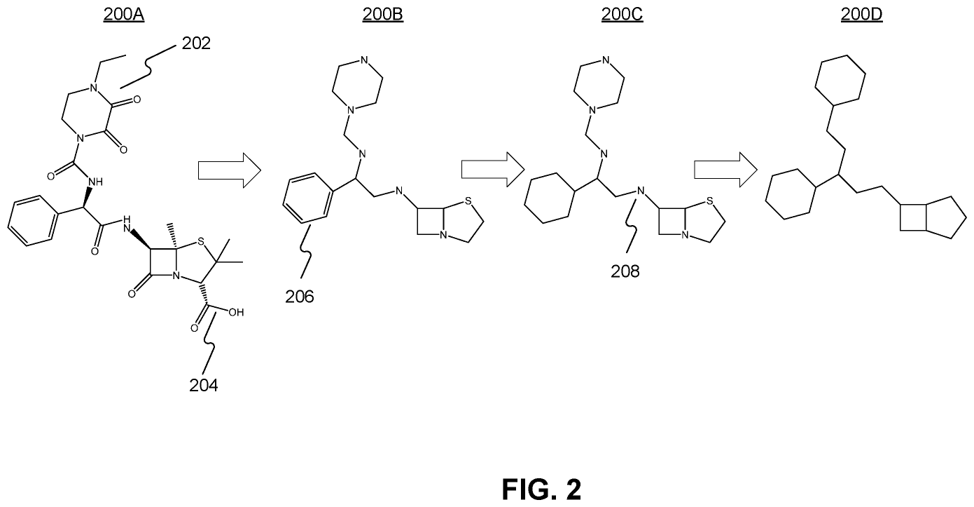

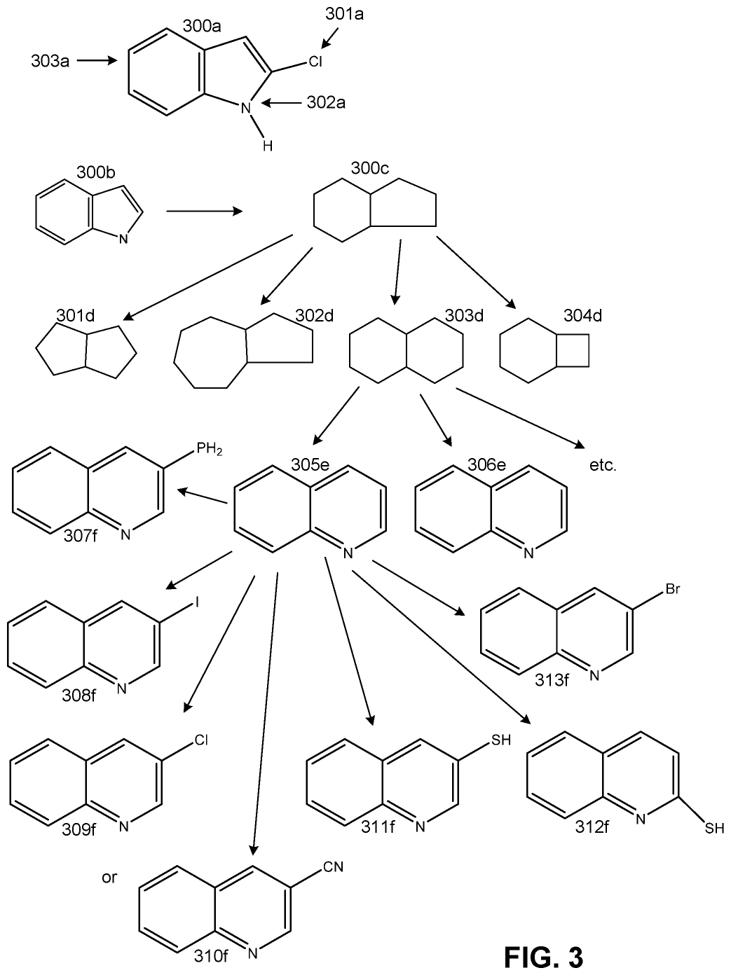

Client device 102 may be configured to receive input from a user, e.g., a query compound. As described below with respect to FIG. 2, supervised learning engine 108 may receive the query compound and generate a query graph framework based on the query compound. Supervised learning engine 108 may also generate one or more derivative graph frameworks, as described below with respect to FIG. 3. Supervised learning engine 108 may then query database 110 for the query graph framework and one or more derivative graph frameworks. As described above, the queries will yield a list comprising a set of frameworks with hits in database 110 and a set of frameworks that are not present in database 110. This list may be returned to client device 102 and presented to the user via a graphical user interface displayed by client device 102.

The supervised learning engine 108 may provide users with the ability to identify compounds that are in the hit and open groups, better allowing a patent drafter to draft Markush claims. Once the supervised learning engine runs on a query compound it may identify Markush structures that are novel, open “areas” (e.g., sets of unclaimed or undisclosed structures), thereby allowing the drafter to claim broadly. Compounds found in the hit group may be used to create a competitive landscape, possibly allowing drafters to draft Markush claims broadly without a fear of rejection or such compounds may be used to determine the extent to which a given Markush structure includes known hits as of a specific point in time. Compounds found in the open group can also be used by drafter to ensure the patent's written description contains enough different examples (i.e., the disclosed species) to sufficiently support the full scope of the Markush (i.e., claimed genus).

By way of example, FIG. 2 illustrates an exemplary decomposition of a query compound 300A, consistent with the disclosed embodiments. The engine 108 may remove substituents such as bonded at 202 and 204, resulting in the graph node bond framework representation 200B of query compound 200A. The engine 108 may further remove substituent bonds 206 and represent the query compound 200A as a graph node framework 200C. The graph node framework 200C may be further decomposed by removing specific node requirements such as 208 and thereby represent the query compound 200A as a graph framework 200D. The graph framework 200D may provide a base from which to analyze a Markush group.

Aspects of the disclosed embodiments may include adding a substituent to each of the at least one derivative graph node bond framework. The supervised learning engine may create possible permutations of a query compound by adding a substituent to each derivative graph node bond frameworks. This may result in various permutations or mutations of the query compound which may be members of a Markush group corresponding to the query compound.

By way of example, FIG. 3 is an exemplary query graph framework and derivative graph node bond framework. As depicted in FIG. 3, a query compound may be 3-Chloro-1H-Indole 300a, a substituent compound may be Chloride 301a. The graph node bond framework of the query compound 3-Chloro-1H-Indole 300a may be the fused five 302a and six 303a member ring, together 300b. The query compound may be input as a table, an image, a chemical formula, or any other input recognizable or readable by the supervised learning engine 108. In some embodiments, the query compound may be input as a CAS Registry Number (“CAS RN”), simplified molecular-input line-entry system (“SMILES”) string, International Chemical Identifier (“InChI”), Molecular Query Language (“MQL”), SYBYL line notation, SMILES arbitrary target specification (“SMARTS”), or other language or symbol representing a chemical compound. The input may indicate, to the supervised learning engine, descriptors or features associated with a compound. Descriptors may indicate known, calculated, or predicted physical properties of a compound. Descriptors may additionally indicate properties of elements and the structure of a compound.

The supervised learning engine 108 may create a query graph framework from the query graph node bond framework by removing bonds as demonstrated by compound 300c. The supervised learning engine 108 may create derivative graph node bond frameworks by altering nodes and edges representing substituents as demonstrated in FIG. 3 by compounds 301d, 302d, 303d, and 304d. The engine may either alter the five 302a or six 303a member ring. In this instance, that the supervised learning engine chose to substitute the query compound 3-Chloro-1H-Indole 300a, it could do so by either adding or deleting nodes.

The supervised learning engine 108 may further add a node or edge to the derivative graph node bond frameworks 301d, 302d, 303d, and 304d. In other embodiments, the query compound may eliminate or increase carbons or heteroatoms within itself. The supervised learning engine 108 may also build bonding back into the new derivative frameworks by adding edges to the derivative graph node bond frameworks. There are multiple variations that the supervised learning engine could come up with. These series of variations are known as derivative graph-node-bond frameworks, and can be generated for each derivative graph framework. Some examples of derivative graph-node-bond frameworks for the query compound 3-Chloro-1H-Indole 300a are demonstrated in FIG. 3 as compounds 305e and 306e.

Aspects of the disclosed embodiments may further include identifying, by the supervised learning engine, for each substituent, a series of bioisosteres. Bioisosteres may include chemical substituents or groups with similar physical or chemical properties which produce broadly similar biological properties to another chemical compound. For each substituent, a series of bioisosteres can be identified by the supervised learning engine. For example, in FIG. 3, a substituent 101a, it may be replaced with an identified bioisostere as demonstrated by compounds 307f, 308f, 309f, 310f, 311f, 312f, and 313f.

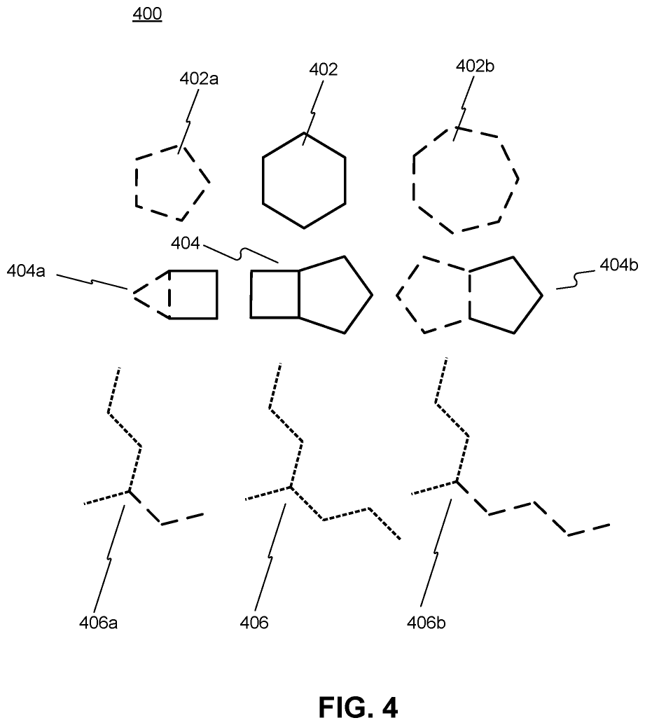

FIG. 4 illustrates possible Markush compounds based on a derivative graph node bond framework. In this example, the supervised learning engine 108 may vary a ring size or linker length of sections of the derivative graph node bond framework 300D representing query compound 300A from FIG. 3. The derivative node bond framework ring size may be represented with solid lines whereas dashed lines may represent possible substituents. The supervised learning engine 108 may subtract an atom from the fused six member ring 402 in order to form a five member ring 402a. Alternatively, the supervised learning engine 108 may add an atom to the fused six member ring 402 in order to form a seven member ring 402b. Additionally, the supervised learning engine 108 may decrease the ring size of 404 in order to form molecule 402a. The ring size of 404 may also be increased in order to form molecule 402b for analysis by the supervised learning engine 108.

Additionally, the length of linker section 406 (represented by dotted lines) may be contracted by one atom, resulting in linker section 406a. The supervised learning engine may also expand the length of linker section 406 by one atom, resulting in linker section 406b. The resulting substituents (402a, 402b, 404a, 404b, 406a, and 406b) may be used in any combination to generate possible members of a Markush group corresponding to the derivative graph node bond framework 200D representing query compound 200A from FIG. 2.

In some embodiments, the supervised learning engine may filter the derivative graph node bond frameworks created which represent compounds. The supervised learning engine 108 may create or refrain from creating a derivative graph node bond framework according to properties or characteristics, such as chemical feasibility of the resulting compound. Filtering may include removing derivative graph node bond frameworks from the analysis by the supervised learning engine based on known, projected, or calculated properties of the compound represented by the derivative graph node bond framework such as chemical feasibility. Chemical feasibility may refer to the possibility, capability, or likelihood of the compound represented by the derivative graph node bond framework existing or being made to exist. Filtering may prevent the supervised learning engine from analyzing compounds that would be impossible to find or make. After the supervised learning engine 108 filters the derivative graph node bond frameworks, a comparison may be run against one or more databases. A database may include a public or private collection of data, as disclosed herein. For example, a Markush database may include Markush compounds or structures publicly disclosed, such as in a printed publication, patent, or patent application. In another example, a chemical registry database may contain organic and inorganic chemical substances, such as alloys, coordination compounds, minerals, mixtures, polymers and salts, and biosequences.

An output list may include graph node bond frameworks or compounds identified as possible members of a Markush group. The output list may indicate a set of graph node bond frameworks or compounds as known. The output list may indicate another set of graph node bond frameworks or compounds as novel. The output list may further indicate derivative graph node bond frameworks which were filtered out from the analysis, for example due to chemical infeasibility. The output list may be received by a client device over a network from a processing device which includes the supervised learning engine and one or more databases or access to one or more databases.

In some embodiments the set of known compounds is determined by comparing properties of the at least one derivative graph node frameworks against the database of known compound properties. On the other hand, if one of the derivative graph-node-bond frameworks does not hit it will be put into an open group. The open group may contain compounds that have not been publicly disclosed in journal articles or published patent applications.

In some embodiments the supervised learning engine 108 may rank the set of novel compounds according to a synthesizability index associated with the set of novel compounds. A synthesizability index may include a variable representing the effort, cost, time, or other variable indicating the difficulty of making or producing an identified compound. Some compounds may be identified or formed using the supervised learning engine but may be difficult to physically produce. Ranking according to the synthesizability index may include arranging the set of novel compounds according to a variable indicating synthesizability of the identified compound. Additionally or alternatively, the supervised learning engine may rank the set of novel compounds according to any known, calculated, or predicted properties or activities associated with each compound in the set of novel compounds.

In further embodiments the supervised learning engine 108 may monitor an identified white space. A white space may include an area identified as an unoccupied region of chemical space. The supervised learning engine may monitor a white space by periodically comparing the set of novel compounds against known compounds. The supervised learning engine 108 may store iterations of the set of novel compounds and corresponding metadata such as a date and location of the disclosure in database 110. The supervised learning engine 108 may compare iterations of the set of novel compounds and output a list or alert when iterations of the set of novel compounds differ. The catalogue may be ranked according the metadata such as location of the disclosure.

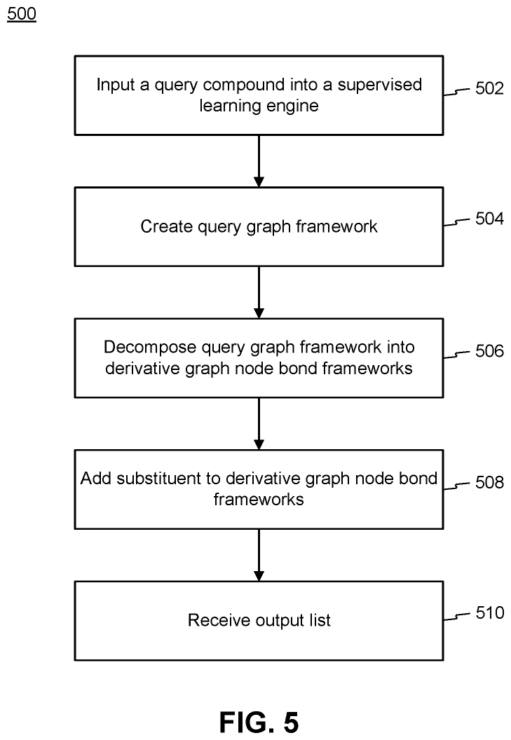

FIG. 5 is a flow diagram of an exemplary method of implementing a supervised learning engine 108 to conduct a prior art and novel Markush landscaping search. The method may begin at step 502 by inputting a query compound into the supervised learning engine. The input may include a table, an image, a chemical formula, or any other input recognizable or readable by the supervised learning engine 108. In some embodiments, the query compound may be input as a CAS Registry Number (“CAS RN”), simplified molecular-input line-entry system (“SMILES”) string, International Chemical Identifier (“InChI”), Molecular Query Language (“MQL”), SYBYL line notation, SMILES arbitrary target specification (“SMARTS”), or other language or symbol representing a chemical compound. The input may be stored by the supervised learning engine 108 as a query graph framework.

At step 504, the supervised learning engine 108 may create a query graph framework. The supervised learning engine 108 may create a query graph framework by storing the query compound using a node-edge graph framework. A node-edge graph framework may represent data as a nodes and connections to other data as edges. For chemical compounds, nodes may represent atoms and a corresponding location of the atom. The edges may represent bonds between atoms.

At step 506, the supervised learning engine 108 may decompose the query graph framework into derivative graph node bond frameworks. The supervised learning engine 108 may divide the query graph framework into sections. The supervised learning engine 108 may either add a node, subtract a node, or maintain the current number of nodes, simulating permutations and mutations to the query compound. The derivative graph node bond framework may represent different pieces or decomposed sections of the query compound. The supervised learning engine 108 may recursively divide the derivative graph node bond framework until the resulting derivative graph node bond framework represents a substituent molecule or atom.

At step 508, supervised learning engine 108 may subtract or add one or more substituents to the derivative graph node bond frameworks to produce a representation of a possible compound within a Markush group corresponding to the query compound. The supervised learning engine 108 may run a comparison if the identified possible compounds against a database of compounds. The supervised learning engine 108 may compare each identified possible compound against graph node framework representations of known compounds. For example, the supervised learning engine 108 may compare simplified molecular-input line-entry system (“SMILES”) string, International Chemical Identifier (“InChI”), Molecular Query Language (“MQL”), SYBYL line notation, SMILES arbitrary target specification (“SMARTS”), or other language or symbolic representations of each identified possible compound against representations of known compounds. A known compound may include compounds disclosed or stored in a database. When the supervised learning engine 108 identifies a match between the identified possible compound and a known compound, the supervised learning engine may store the identified possible compound in a set of hits or known compounds. If the supervised learning engine 108 does not identify a match between the identified possible compound and a known compound, then the supervised learning engine may store the identified possible compound in a set of open or novel compounds.

At step 510, supervised learning engine 108 may send client device 102 an output list. An output list may include a set of novel compounds and a set of known compounds. A set of known compounds may include compound identified when one of the graph node bond frameworks hits against a known compound or Markush structure that has been publicly disclosed, the graph-node-bond framework may be moved to a hit category. The hit category may contain compounds that have already been publicly disclosed and may be used in a prior art or landscaping analysis. For example, in the instance that the supervised learning engine uses the derivative graph-node-bond framework 303d, it may then filter derivative node bond frameworks 305e or 306e by chemical feasibility. In a situation where derivative-node-bond framework 306e is not chemically feasible, the engine would discard derivative node bond framework 306e and create and analyze a chemically feasible derivative-node-bond framework, such as 305e.

A set of novel compounds may include graph node bond frameworks identified but not placed in the hit category. These novel compounds may be indicated as an open group. For each derivative graph-node-bond framework put into the open group, each substituent from the query compound and the series of bioisosteres generated may be used to enumerate novel compounds by combinatorial addition of these substituents at locations mapped to the original query compound, as demonstrated in FIG. 3 by compounds 307f, 308f, 309f, 310f, 311f, 312f, and 313f.

The disclosed systems and methods may be used to evaluate prior art and its similarities to one or more documents such as new patent applications. The disclosed systems and methods may provide increased accuracy over prior systems, which are inefficient and require human intervention at one or more steps.

In one embodiment, systems and methods consistent with the present disclosure may receive a patent application or other document as an input and output related prior art results and/or other related documents. Such systems and methods may be used, for example, to find prior art related to a newly submitted patent application. In other embodiments, the described systems and methods may be used to perform related art searches prior to submitting a patent application or may be used to assist in freedom-to-operate analyses.

The systems and methods described herein may be used by, for example, commercial, government, or academic entities, including but not limited to scientists, intellectual property professionals, legal professionals, business professionals, patent-office examiners, regulatory bodies, and academics. In an embodiment, the system may enable a user to perform a similarity search between published patent applications (or other documents) and a new patent application (or other document). In some embodiments, the system may output a document determined to be most similar to the inputted document or a list of similar documents ranked based on their similarity to the inputted document.

It is to be understood that the disclosed embodiments are not necessarily limited in their application to the details of construction and the arrangement of the components and/or methods set forth in the following description and/or illustrated in the drawings and/or the examples. The disclosed embodiments are capable of variations, or of being practiced or carried out in various ways.

The disclosed embodiments may be implemented in a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowcharts and block diagrams in the Figures illustrate the architecture, functionality, and operation of possible implementations of systems, methods and computer program products according to various embodiments of the present invention. In this regard, each block in the flowcharts or block diagrams may represent a software program, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A computer-implemented system, comprising:
a memory device storing a set of instructions; and
at least one processor executing the set of instructions to perform a method, the method comprising:
inputting a query compound into a supervised learning engine;
creating, by the supervised learning engine, a query graph framework;
decomposing, by the supervised learning engine, the query graph framework into at least one derivative graph node bond framework;
adding a substituent to each of the at least one derivative graph node bond frameworks; and
receiving, from the engine, an output list comprising a set of novel compounds and a set of known compounds.

2. The system of claim 1, the method further comprising:
identifying, by the supervised learning engine, for each substituent, a series of bioisosteres.

3. The system of claim 1, wherein the set of novel compounds is determined by comparing properties of the at least one derivative graph node frameworks against a database of known compound properties.

4. The system of claim 3, wherein the set of known compounds is determined by comparing properties of the at least one derivative graph node frameworks against the database of known compound properties.

5. The system of claim 1, wherein decomposing the query graph framework comprises at least one of subtracting a node or adding a node.

6. The system of claim 1, the method further comprising:
filtering the at least one derivative graph node bond framework by chemical feasibility.

7. The system of claim 1, wherein the set of novel compounds is determined by comparing the at least one derivative graph node frameworks against a database of publicly disclosed compounds.

8. The system of claim 7, wherein the set of known compounds is determined by comparing the at least one derivative graph node frameworks against the database of publicly disclosed compounds.

9. The system of claim 8, wherein the database of publicly disclosed compounds comprises patent documents.

10. The system of claim 1, wherein the output list ranks the set of novel compounds according to at least one of a synthesizability index, a property, or an activity associated with the set of novel compounds.

11. A computer-implemented method comprising:
inputting a query compound into a supervised learning engine;
creating, by the supervised learning engine, a query graph framework;
decomposing, by the supervised learning engine, the query graph framework into at least one derivative graph node bond frameworks;
adding a substituent to each of the at least one derivative graph node bond frameworks; and
receiving, from the engine, an output list comprising a set of novel compounds and a set of known compounds

12. The method of claim 11, the method further comprising:
identifying, by the supervised learning engine, for each substituent, a series of bioisosteres.

13. The method of claim 11, wherein the set of novel compounds is determined by comparing properties of the at least one derivative graph node frameworks against a database of known compound properties.

14. The method of claim 13, wherein the set of known compounds is determined by comparing properties of the at least one derivative graph node frameworks against the database of known compound properties.

15. The method of claim 11, wherein decomposing the query graph framework comprises at least one of subtracting a node or adding a node.

16. The method of claim 11, the method further comprising:
filtering the at least one derivative graph node bond framework by chemical feasibility.

17. The method of claim 11, wherein the set of novel compounds is determined by comparing the at least one derivative graph node frameworks against a database of publicly disclosed compounds.

18. The method of claim 17, wherein the set of known compounds is determined by comparing the at least one derivative graph node frameworks against the database of publicly disclosed compounds.

19. The method of claim 18, wherein the database of publicly disclosed compounds comprises patent documents.

20. The method of claim 11, wherein the output list ranks the set of novel compounds according to at least one of a synthesizability index, a property, or an activity associated with the set of novel compounds.
